(19) United States
(12) Patent Application Publication
(10) Pub. No.: US 2004/0059369 A1
(43) Pub. Date: Mar. 25, 2004

(54) Catheter and guide wire exchange system

(76) Inventors: Niall Duffy, Galway (IE); Gerry Clarke, Galway (IE); Noel Coyle, Galway (IE); Patrick J. Duane, Galway (IE); John MacNamara, Galway (IE); Ashish Varma, Galway (IE); P.J. Carmody, Galway (IE); David Quinn, Galway (IE)

Correspondence Address: MEDTRONIC AVE, INC., 3576 UNOCAL PLACE, SANTA ROSA, CA, 95403, US

(21) Appl. No.: 10/251,578

(22) Filed: Sep. 20, 2002

(51) Int. Cl. A61M 29/00

(52) U.S. Cl. 606/194

(57) ABSTRACT

A catheter and guide wire exchange system including a catheter having a guide wire lumen with a guide way extending along the length of a stiffened proximal shaft portion, and a guide member slidably disposed about the proximal shaft for directing a guide wire into or out of the guide way and the guide wire lumen. The proximal shaft may be slid through the guide member so that the guide wire is contained within the guide wire lumen distal to the guide member and with the guide wire and catheter being separated proximal of the guide member.

### FIELD OF THE INVENTION

The present invention relates to catheters used in the vascular system and more particularly to a system for facilitating exchange of such catheters and guide wires, and for transporting such catheters and guide wires to selected sites within a patient.

### BACKGROUND OF THE INVENTION

Catheters are inserted to various locations within a patient for a wide variety of purposes and medical procedures. For example only, one type of catheter is used in percutaneous catheter intervention (PCI) for the treatment of a vascular constriction termed a stenosis. In this instance, the catheter has a distally mounted balloon that can be placed, in a deflated condition, within the stenosis, and then inflated to dilate the narrowed lumen of the blood vessel. Such balloon dilation therapy is generally named percutaneous transluminal angioplasty (PTA). The designation PTCA, for percutaneous transluminal coronary angioplasty, is used when the treatment is more specifically employed in vessels of the heart. PTCA is used to open coronary arteries that have been occluded by a build-up of cholesterol fats or atherosclerotic plaque. The balloon at the distal end of the catheter is inflated, causing the site of the stenosis to widen.

The dilation of the occlusion, however, can form flaps, fissures and dissections, which may result in reclosure of the dilated vessel or even perforations in the vessel wall. Implantation of a stent can provide support for such flaps and dissections and thereby prevent reclosure of the vessel or provide a patch repair for a perforated vessel wall until corrective surgery can be performed. A stent is typically a cylindrically shaped device formed from wire(s) or a metal tube and is intended to act as a permanent prosthesis. A stent is deployed in a body lumen from a radially compressed configuration into a radially expanded configuration that allows it to contact and support a body lumen. A stent can be implanted during an angioplasty procedure by using a balloon catheter bearing a compressed stent that has been loaded onto the balloon. The stent radially expands as the balloon is inflated, forcing the stent into contact with the body lumen, thereby forming a supporting relationship with the lumen walls. Alternatively, self-expanding stents may be deployed with a sheath-based delivery catheter. Deployment is effected after the stent has been introduced percutaneously, transported transluminally and positioned at a desired location by the delivery catheter. In addition to angioplasty and stenting procedures, other therapeutic procedures require use of a delivery catheter, such as drug delivery, filters, occlusion devices, diagnostic devices and radiation treatment.

Typically, the placement of such therapeutic delivery catheters involves the use of a guide wire, which may be inserted into the patient's vasculature through the skin, and advanced to the location of the treatment site. The delivery catheter, which has a lumen adapted to receive the guide wire, then is advanced over the guide wire. Alternatively, the guide wire and the delivery catheter may be advanced together, with the guide wire protruding from the distal end of the delivery catheter. In either case, the guide wire serves to guide the delivery catheter to the location to be treated.

To treat small diameter vessels remote from the entry point into the patient, a guiding catheter is used to span the distance. For example, in PTCA or stent delivery, a guiding catheter 10 is typically inserted into a large artery 12 near the patient's groin, and then advanced toward the heart 14 to the entry opening, or ostium, of the diseased coronary artery as shown in FIGS. 1A and 1B. The guiding catheter 10 provides a tubular conduit through which catheters and guide wires, such as catheter 16 and guide wire 18, can be passed from outside the patient through hemostatic valve 34 to the vessel being treated.

There are four general types of catheters: “over-the-wire” (OTW) catheters, “over-the-wire catheters with short wire capability” (OTW/SW) such as disclosed in U.S. Pat. No. 4,998,356 (Crittenden, et al.) and co-pending application U.S. Ser. No. 10/116,234, which is incorporated by reference herein, “rapid exchange” catheters and “fixed wire” or “a balloon on a wire” catheters. OTW and rapid exchange catheters require use of a guide wire separate from the catheter while a fixed wire or balloon on a wire catheter has an integral guide wire. An OTW catheter comprises a guide wire lumen that extends the entire length of the catheter. The guide wire is disposed entirely within the catheter guide wire lumen except for distal and proximal portions of the guide wire, which extend beyond the distal and proximal ends of the catheter respectively. An OTW/SW catheter has an over-the-wire configuration while the catheter is within the patient's body. Thus, the guide wire is disposed entirely within the catheter guide wire lumen, except for the distal and proximal portion of the guide wire, which extend beyond the distal and proximal ends of the catheter respectively when it is fully inserted into the patient.

OTW and OTW/SW catheters have many advantages traceable to the presence of the full length guide wire lumen, such as good stiffness and pushabilty for readily advancing the catheter through the tortuous vasculature and across tight stenosis. The full-length guide wire lumen permits removable and replacement of a guide wire in an indwelling catheter, as may be required to alter the shape of the guide wire tip. It is also sometimes desirable to exchange one guide wire for another guide wire having a different stiffness. For example, a relatively soft, or flexible guide wire may prove to be suitable for guiding a PTCA catheter through a particular tortuous anatomy, whereas following up with a stent delivery catheter through the same vasculature region may require a guide wire that is relatively stiffer.

Traditional over-the-wire catheters do suffer some shortcomings, however. For example, it often becomes necessary, in the performance of a PCI, to exchange one indwelling catheter for another catheter. In order to maintain a guide wire in position while withdrawing the catheter, the guide wire must be gripped at its proximal end to prevent it from being pulled out of the blood vessel with the catheter. For example, a PTCA catheter, which may typically be on the order of 135 centimeters long, is longer than the proximal portion of the standard guide wire that protrudes out of patient. Therefore, exchanging an over-the-wire PTCA catheter requires an exchange guide wire of about 300 centimeters long, whereas a standard guide wire is about 165 centimeters long.

In one type of over-the-wire catheter exchange, the standard length guide wire first is removed from the lumen of the indwelling catheter. Then, a longer exchange guide wire is passed through the catheter to replace the original wire. Next, while holding the exchange guide wire by its proximal end to control its position in the patient, the catheter is withdrawn proximally from the blood vessel over the exchange guide wire. After the first catheter has been removed, the next OTW catheter is threaded onto the proximal end of the exchange guide wire and is advanced along the exchange guide wire, through the guiding catheter, and into the patient's blood vessels until the distal end of the catheter is at the desired location. The exchange guide wire may be left in place or it may be exchanged for a shorter, conventional-length guide wire. In an alternative type of catheter exchange procedure, the length of the initial guide wire may be extended by way of a guide wire extension apparatus. Regardless of which exchange process is used, the very long exchange guide wire is awkward to handle, thus requiring at least two operators to perform the procedure.

An OTW catheter designed to eliminate the need for guide wire extensions or exchange wires is disclosed in U.S. Pat. No. 4,988,356 (Crittenden et al.). This OTW/SW catheter includes a catheter shaft having a cut that extends longitudinally between the proximal end and the distal end of the catheter and that extends radially from the catheter shaft outer surface to the guide wire lumen. A guide member slidably coupled to the catheter shaft functions to open the cut such that the guide wire may extend transversely into or out of the cut at any location along its length. By moving the guide member, the effective over-the-wire length of the OTW/SW catheter is adjustable.

When using the OTW/SW catheter, the guide wire is maneuvered through the patient's vascular system such that the distal end of the guide wire is positioned across the treatment site. With the guide member positioned near the distal end of the catheter, the proximal end of the guide wire is threaded into the guide wire lumen opening at the distal end of the catheter and through the guide member such that the proximal end of the guide wire protrudes out the proximal end of the guide member. By securing the guide member and the proximal end of the guide wire in a fixed position, the catheter may then be transported over the guide wire by advancing the catheter toward the guide member. In doing so, the catheter advances through the guide member such that the guide wire lumen envelops the guide wire as the catheter is advanced into the patient's vasculature. In a PTCA embodiment, the OTW/SW catheter may be advanced over the guide wire in this manner until the distal end of the catheter having the dilatation balloon is positioned within the stenosis and essentially the entire length of the guide wire is encompassed within the guide wire lumen.

Furthermore, the indwelling OTW/SW catheter may be exchanged with another catheter by reversing the operation described above. To this end, the indwelling catheter may be removed by withdrawing the proximal end of the catheter from the patient while holding the proximal end of the guide wire and the guide member in a fixed position. When the catheter has been withdrawn to the point where the distal end of the cut has reached the guide member, the distal portion of the catheter over the guide wire is of a sufficiently short length that the catheter may be drawn over the proximal end of the guide wire without releasing control of the guide wire or disturbing its position within the patient. After the catheter has been removed, another OTW/SW catheter may be threaded onto the guide wire and advanced over the guide wire in the same manner described above with regard to the OTW/SW catheter. The OTW/SW catheter not only permits catheter exchange without the use of the very long exchange guide wire and without requiring withdrawal of the initially placed guide wire, but it also overcomes many of the other difficulties discussed in association with rapid exchange catheters described below.

Rapid exchange catheters developed in attempt to eliminate the need for a guide wire extension or exchange wires. Catheters of this type are formed so that the guide wire is located outside of the catheter except for a short guide wire lumen that extends within only a comparatively short distal segment of the catheter. The rapid exchange catheter's proximal exit port for the guide wire is typically located about 5 cm (2.0 in) to 30 cm (11.8 in) proximal to the catheter's distal end. In use, the guide wire is placed initially in the patient's vascular system. The distal segment of the rapid exchange catheter then is threaded onto the wire. The catheter can be advanced alongside the guide wire with its distal segment being attached to and guided along the guide wire. The rapid exchange catheter can be removed and exchanged for another rapid exchange catheter without the use of a very long exchange guide wire and without requiring withdrawal of the initially placed guide wire.

A difficulty associated with rapid exchange catheters is that it is not possible to exchange guide wires in an indwelling rapid exchange catheter, as can be done advantageously with OTW catheters. A guide wire can be withdrawn, sometimes unintentionally, from the proximal guide wire port, thus derailing an indwelling rapid exchange catheter. However, neither the first guide wire, nor a replacement guide wire, can be directed back into the catheter's proximal guide wire port, which is hidden remotely in the guiding catheter within the patient.

Guide wires are commonly back loaded into the delivery catheter. In this operation, the guide wire proximal end is inserted into the distal tip of the catheter. It is pushed through the catheter until it extends out of the proximal guide wire exit. In a traditional over-the-wire catheter the proximal guide wire exit is the proximal end of the catheter through its inflation luer. The rapid exchange proximal guide wire exit is the termination of the short guide wire tube a few centimeters or typically 25 centimeters beyond the distal tip of the catheter. In the OTW/SW catheter, the proximal guide wire exit is through the guide member positioned on the proximal shaft of the catheter. As an alternative to back loading a guide wire into the delivery system, a guide wire may also be front-loaded. In a front-loading operation, the distal tip of the guide wire is inserted into the guide wire lumen on the proximal shaft and pushed through until it exits the distal tip of the delivery catheter. A front-loading operation is possible with OTW and OTW/SW catheters if the guide wire will be exchanged during procedures. A front loading operation is not used with a rapid exchange catheter since the guide wire cannot be exchanged while the catheter is inserted into the patient. With a rapid exchange catheter, the insertion of the distal tip into the proximal end of the guide wire lumen is pure chance due to the fact that the proximal end is typically 125 centimeters from the exit location of the catheter from the patient at the femoral artery in the groin.

The guide member of the OTW/SW catheter is used for both advancement of the catheter into the patient and for exchanging the guide wire during the procedure without removing the catheter. Thus, the present invention is directed towards various embodiments of the guide member that optimize the versatility of the dual function of the guide member.

### SUMMARY OF THE INVENTION

The present invention is a guide member for an OTW/SW catheter and guide wire exchange system. The OTW/SW catheter and guide wire exchange system comprises an elongate flexible catheter having proximal and distal ends and first and second lumens extending there through. The first lumen is open at the shaft distal end and is sized and shaped to sizably receive a guide wire. The catheter has a bitumen proximal shaft and a coaxial distal shaft. The guide member is mounted on the catheter proximal shaft and is received in a guide way formed from a longitudinal cut in a catheter proximal shaft to enable transverse access to the first lumen through the elongate flexible catheter. The guide way extends along a major portion of the length of the proximal shaft from a location adjacent to the proximal end of the catheter to a location proximal of the proximal shaft distal end. An enlarged stop is located on the exterior of the proximal shaft distal end. The guide member cannot travel distally past the stop. A balloon is mounted about catheter distal segment, with the balloon being in fluid communication with the second lumen.

The guide member has a catheter passageway that extends longitudinally through the guide member and a guide wire passageway for slidably receiving a guide wire therethrough. The guide member includes a keel that slides along the guide way to assist in merging the guide wire into the first lumen as the catheter shaft is moved through the catheter passageway. Conversely, the guide member can be used for separating the guide wire and catheter by guiding the guide wire out of the guide wire lumen through the guide way.

### BRIEF DESCRIPTION OF THE DRAWINGS

These and other features, aspects and advantages of the present invention will become better understood with reference to the following description, appended claims, and accompanying drawings where:

FIG. 1A is a diagrammatic illustration of a patient showing the manner in which a balloon catheter is advanced from the femoral artery through the aorta and into the patient's heart;

FIG. 1B is an enlarged portion of FIG. 1A showing the present invention positioned with the guide catheter and extending into the femoral artery;

FIG. 2 is an illustration of a OTW/SW catheter and guide wire in an assembled configuration;

FIG. 2A is a cross-section taken along line A-A of FIG. 5;

FIG. 2B is a cross-section taken along line B-B of FIG. 5;

FIG. 2C is a cross-section taken along line C-C of FIG. 5;

FIG. 3 is a cross sectional view of the intersection of the guide member, guide wire and proximal shaft of a OTW/SW catheter;

FIGS. 4-6 are perspective views of the guide member of the present invention showing certain internal features in hidden line;

FIG. 7 is a partially sectioned view of a proximal shaft and guide member of a OTW/SW catheter;

FIG. 8 is a partially sectioned view of a proximal shaft and guide member of a OTW/SW catheter;

FIG. 9 is an illustration of the insertion and removal of a OTW/SW catheter with respect to a hemostatic valve;

FIG. 10 is an alternative embodiment of a guide member of the present invention.

### DETAILED DESCRIPTION OF THE INVENTION

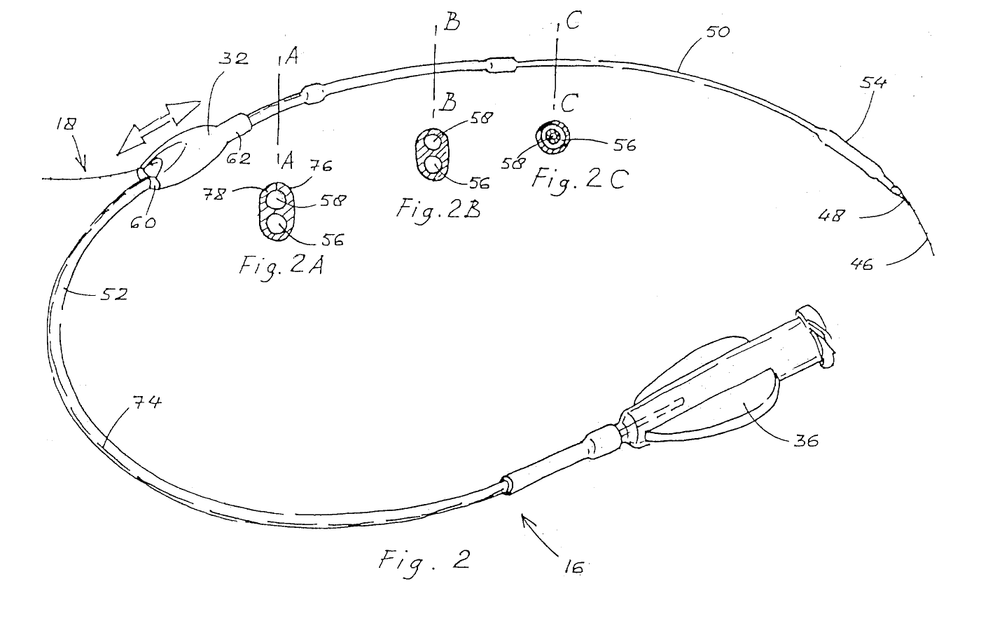

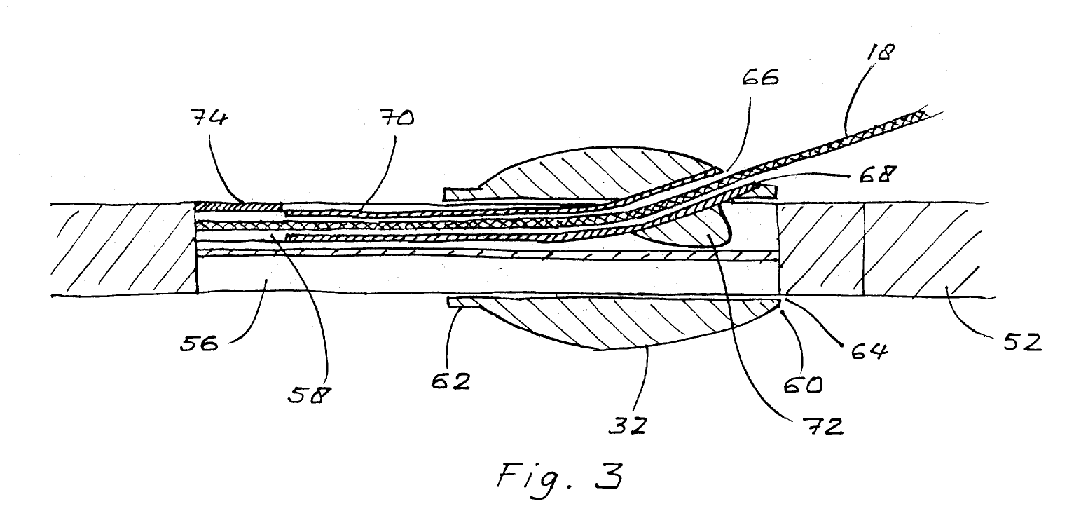

The present invention is a guide member for OTW/SW catheter 16 shown in FIGS. 2-2C. OTW/SW catheter 16 includes guide member 32 with guide wire 18 illustrated as extending through guide member 32. Guide member 32 serves as a juncture in which the catheter 16 and guide wire 18 may be merged or separated so that the portion of guide wire 18 which extends proximally of guide member 32 (to the left as seen in FIG. 2) is separated from catheter 16 and the portion of guide wire 18 which is located distally of guide member 32 (to the right as seen in FIG. 2) is contained and housed within catheter 32 except for distal end 46 of guide wire 18 which may protrude distally out of distal end 48 of catheter 16.

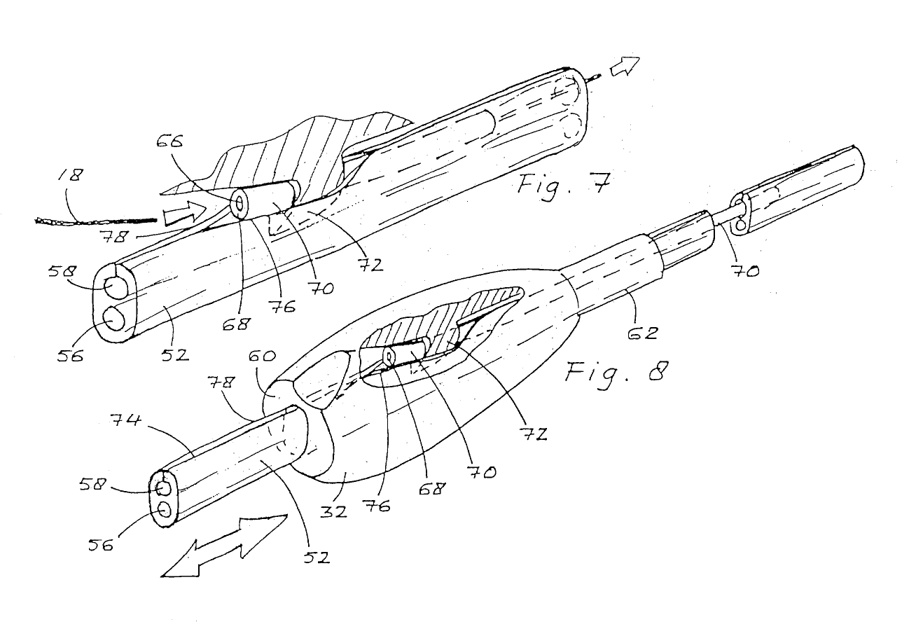

Catheter 16 includes an elongate, flexible, cylindrical main body, which may be formed from an extruded plastic material such as, for example, polyethylene or polyethylene block amide (PEBA) copolymer. Catheter 16 has a distal shaft 50 and a proximal shaft 52. In the embodiment shown in FIG. 5, catheter 16 is a delivery catheter, such as for PTCA or stent delivery, having balloon 54 mounted around the catheter body near catheter distal end 48. Balloon 54 may be inflated and deflated through inflation lumen 56 formed through the body of the catheter 16. Inflation lumen 56 extends from the proximal end of catheter 16, where it communicates with fitting 36 and extends the length of catheter 16, terminating in communication with the interior of balloon 54. Fitting 36 may be connected to a suitable source of pressurized fluid or a partial vacuum (not shown) to inflate or deflate balloon 48. Catheter 16 includes another lumen, indicated at 58, which is intended to receive guide wire 18. Guide wire lumen 58 extends the full length of catheter 16, terminating at distal end 48 and proximal fitting 36.

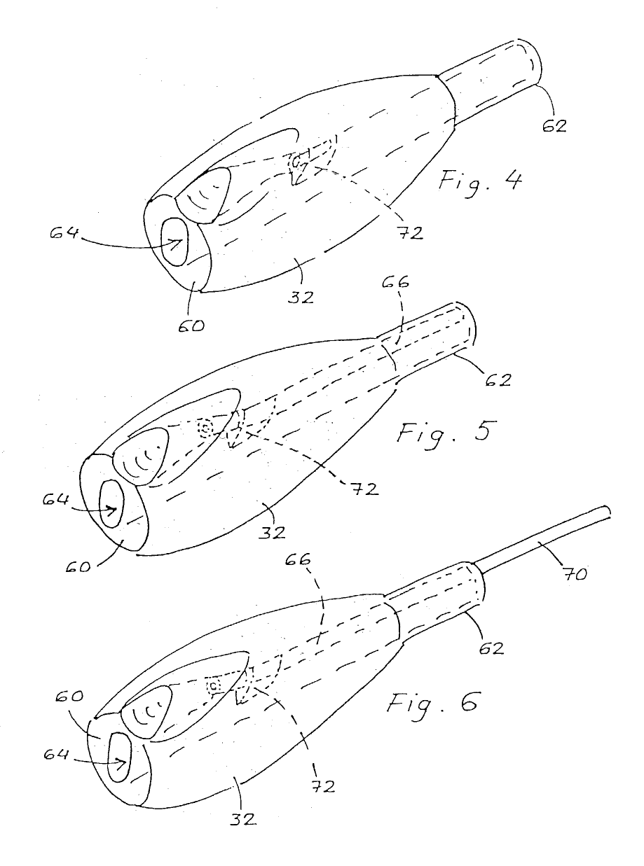

Guide member 32 has proximal and distal ends 60 and 62 as shown in FIGS. 2 and 4-10. Catheter passageway 64 extends longitudinally in a generally straight line from guide member proximal end 60 to guide member distal end 62. Guide wire passageway 66 extends distally from end 68 of tube 70, through tube 70 and into guide wire lumen 58. The length of tube 70 may vary however, it preferably extends through guide wire lumen 58 past the distal end 62 of guide member 32 as shown in FIGS. 3, 6, 8 and 10.

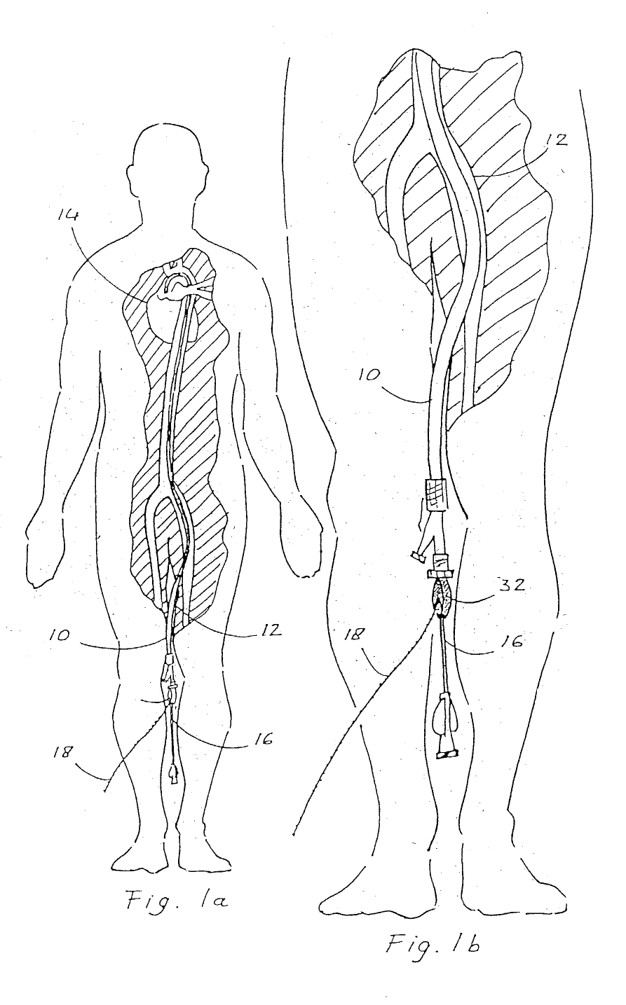

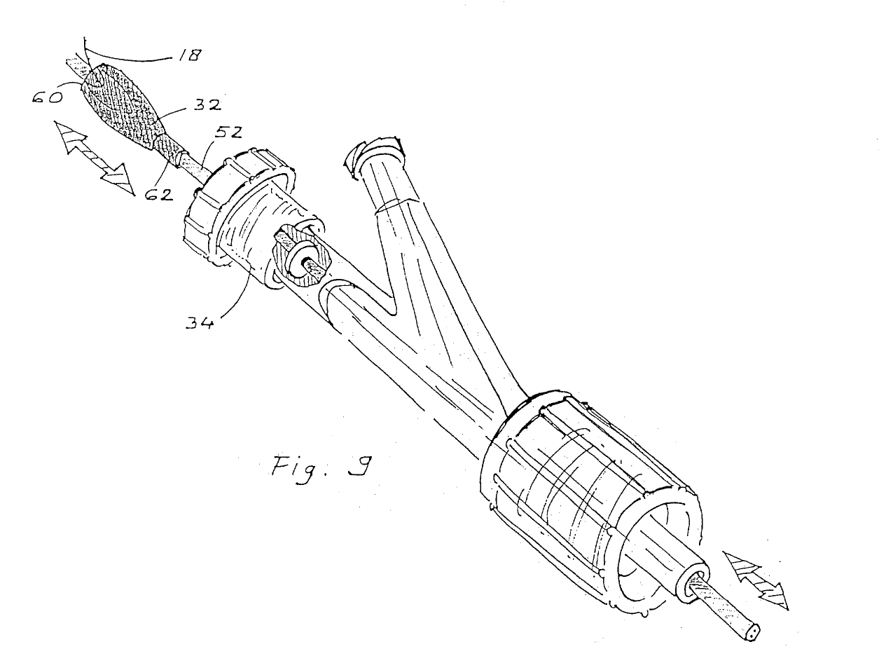

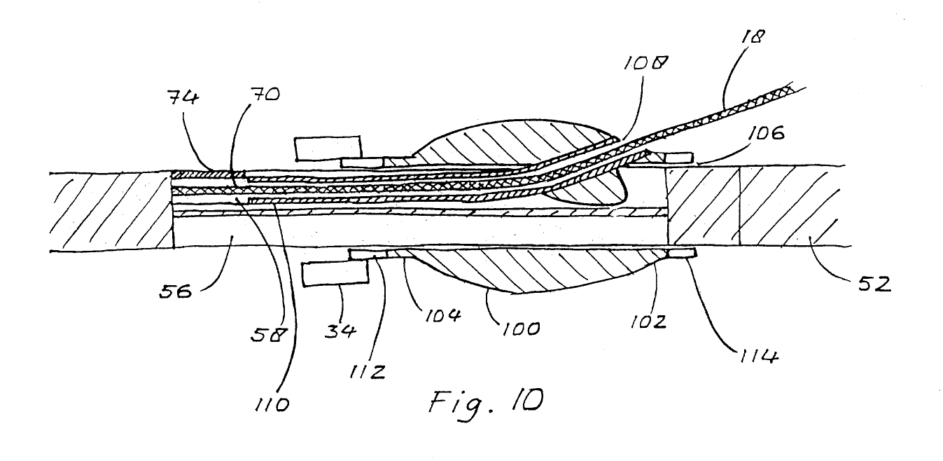

Catheter 16 is shown extending through guide member 32 in FIGS. 2, 3, 7, 8 and 9. Catheter proximal shaft 52 extends through catheter passageway 64, engaging keel 72, which extends through guide way 74 in catheter 16 to spread flaps 70 and 72 apart as indicated in FIGS. 7 and 8. Guide wire 18 may extend through guide wire passageway 68 of tube 70 that enters guide wire lumen 58 through spread-apart flaps 76 and 78. During advancement of catheter 16 through guide member 32, flaps 76 and 78 draw together under the influence of the inherent resiliency of the catheter body to close guide way 74, thus enclosing guide wire 18 within guide wire lumen 58. Guide wire 18 is contained within guide wire lumen 58 from guide member 32 to catheter distal end 48. In an alternative maneuver, guide wire 18 may be inserted or removed through guide wire passageway 66, while guide member 32 is held stationary with respect to catheter 16 as shown by the arrow in FIG. 7. In this fashion, guide wire 18 can be exchanged within catheter 16. In yet another type of manipulation, guide wire 18 and guide member 32 can be held relatively still while catheter shaft 52 is moved, thus bringing guide wire 18 and catheter 16 apart or together, depending on which direction shaft 52 is moved as indicated by the arrows in FIGS. 8 and 9. In use, guide member 32 will be adjacent the hemostatic valve 34 as shown in FIGS. 1A, 1B and 9.

An alternative guide member embodiment is shown in FIG. 10. Guide member 100 has a proximal end 102 and distal end 104 with catheter passageway 106 and guie wire passageway 108 extending through tube 110. Nose 110 extends from distal end 104 and is preferably a hypotube. Additionally, guide member 100 includes a strain relief boss 114 at its proximal end 106. Guide member 32 may also include a proximal or distal strain relief. These strain reliefs assist in keeping guide member 32 or 100 aligned with catheter passageway during operation. Furthermore, in use, hemostatic valve maybe closed down onto the nose 110 and still allow free movement of shaft 52 through guide member 100.

While the invention has been particularly shown and described with reference to the preferred embodiments thereof, it will be understood by those skilled in the art that various changes in form and detail may be made there in without departing from the spirit and scope of the invention.

What is claimed is:

1. A catheter and guide wire exchange system comprising:
an elongate flexible catheter shaft having proximal and distal shafts and first and second lumens extending there through, the first lumen being open at the shaft distal end and being sized and shaped to slidably receive a guide wire;
a longitudinal guide way formed in the proximal shaft to enable transverse access to the first lumen through the proximal shaft, the guide way extending along a major portion of the length of the proximal shaft from a location adjacent a proximal end of the proximal shaft to a distal terminal end proximal of a distal end of the proximal shaft, thereby defining an uncut distal segment of the proximal shaft;
a balloon mounted about a distal segment of the distal shaft, the balloon being in fluid communication with the second lumen; and
a guide member mounted on the proximal shaft and having a catheter passageway extending there through for slidably receiving the catheter shaft and a guide wire passageway for slidably receiving the guide wire for merging the guide wire and the catheter by guiding the guide wire transversely through the guide way and into the first lumen and for separating the guide wire and catheter by guiding the guide wire transversely out of the first lumen through said guide way, the guide member including a keel for tracking the guide way.
